(19) United States
(12) Patent Application Publication
(10) Pub. No.: US 2012/0107860 A1
(43) Pub. Date: May 3, 2012

(54) CELL SORTING APPARATUS, CELL SORTING CHIP, AND CELL SORTING METHOD

(76) Inventor: Yoichi Katsumoto, Tokyo (JP)

(21) Appl. No.: 13/272,942

(22) Filed: Oct. 13, 2011

(30) Foreign Application Priority Data
Oct. 29, 2010 (JP) P2010-244004

(51) Int. Cl. C12Q 1/02; C12M 1/34

(52) U.S. Cl. 435/29; 435/287.1

(57) ABSTRACT

Disclosed herein is a cell sorting apparatus, including: a branch portion branching a flow path in which a fluid containing therein cells flows into a first branch flow path, and a second branch flow path; a coupling portion coupling the first branch flow path and the second branch flow path to each other; and a flowing-out portion causing liquids flowing in the first branch flow path and the second branch flow path coupled to each other by the coupling portion, respectively, to flow out to an outside.

### CROSS REFERENCES TO RELATED APPLICATIONS

The present application claims priority to Japanese Patent Application No. 2010-244004 filed on Oct. 29, 2010, the disclosure of which is incorporated herein by reference.

### BACKGROUND

The present disclosure relates to a cell sorting apparatus, a cell sorting chip, and a cell sorting method for sorting desired cells.

A fluorescence flow cytometer or a cell sorter is known as an apparatus for sorting cells. With such a sorting apparatus, cells are trapped in an air-liquid interface in an outlet port under a suitable vibration condition (in general, a flow rate at an outlet port is several meters/second, and a frequency is several tens of kilohertz) by a circumferential fluid. At the same time, the electric charges are given to each of the cells. Each of the cells flies as a droplet in an air to which a static electric field is applied in a direction corresponding to a quantity of electric charges. Finally, the cells are sorted into sorting containers provided outside a flow path.

This technique is useful when the flow rate is relatively high as with the case described above. However, in the flow cytometer or dielectric cytometer showing the low flow rate, it is difficult for this technique to fulfill the conditions of the droplet and the discharge. For this reason, if anything, it is preferable that the sorting operation is carried out within the flow path having a branch provided therein, and the cells are held in a preceding stage.

With regard to a method of introducing the cells after the branching into the containers, for example, as described in JP-T-2003-507739 (refer to FIG. 38 and the like), there is known a method of drawing a flow containing therein cells to the outside of an apparatus through a pipe.

However, when the flow containing therein the cells is drawn to the outside of the apparatus through the pipe, in general, a liquid sending path becomes long, it takes a lot of time for the flow to reach the container, and a consumption of an amount of liquid is also large.

### SUMMARY

In order to cope with such a situation, the inventors of this technology proposed a cell sorting method of carrying out intra-flow path sorting for cells based on some sort of cell sorting information. In an apparatus realizing this method, a sorting driving force applying portion for applying a driving force to cells in order to sort the cells by changing a path for the cells based on the cell sorting information is provided within a flow path. In addition, this apparatus is provided with two branch flow paths and liquid staying portions. In this case, the two branch flow paths are provided downstream with respect to the sorting driving force applying portion. Also, in the liquid staying portions, the fluids flowing through the two branch flow paths, respectively, are made to stay.

However, in such an apparatus, it is possible that a water head difference or the like in the liquid staying portions are readily generated, and this influence is propagated as a pressure change up to the sorting driving force applying portion through the branch flow paths, thereby impeding the precise sorting of the cells by the sorting driving force applying portion.

The present technology has been made in order to solve the problems described above, and it is therefore desirable to provide a cell sorting apparatus, a cell sorting chip, and a cell sorting method which can precisely sort cells without generating a pressure difference between branch flow paths.

According to an embodiment of the present disclosure, there is provided a cell sorting apparatus including: a branch portion branching a flow path in which a fluid containing therein cells flows into a first branch flow path, and a second branch flow path; a coupling portion coupling the first branch flow path and the second branch flow path to each other; and a flowing-out portion causing liquids flowing in the first branch flow path and the second branch flow path coupled to each other by the coupling portion, respectively, to flow out to an outside.

In the embodiment of the present disclosure, the first branch flow path and the second branch flow path which branch once are coupled to each other again by the coupling portion. Therefore, when the fluids flowing in the first branch flow path and the second branch flow path, respectively, are caused to flow out from the flowing-out portion to the outside, no pressure difference is generated between the first branch flow path and the second branch flow path. Thus, the cells can be precisely sorted.

Preferably, the cell sorting apparatus may further include a cell holding portion provided in the first branch flow path and holding the cells, in which a length of the cell holding portion, and a ratio between an average cross-sectional area of the cell holding portion and an average cross-sectional area of the first branch flow path are set in such a way that a distance by which the cells settle out while the cells pass through the cell holding portion becomes larger than a height of the first branch flow path.

The setting is made in such a way, whereby the desired cells sorted are held by the cell holding portion without flowing into the flowing-out portion. Therefore, the cells sorted can be reliably taken out to the outside from the cell holding portion.

According to another embodiment of the present disclosure, there is provided a cell sorting chip including: a substrate; a flow path which is provided in said substrate and in which a fluid containing therein cells flows; a first branch flow path and a second branch flow path provided in the substrate and branching at the flow path; a cell holding portion provided in the first branch flow path and holding the cells contained in the fluid flowing in the first branch flow path; and a flowing-out portion coupling the first branch flow path and the second branch flow path to each other and causing liquids flowing in the first branch flow path and the second branch flow path, respectively, to flow out to an outside.

In the embodiment of the present disclosure, the first branch flow path and the second branch flow path which branch once are coupled to each other again. Therefore, when the fluids flowing in the first branch flow path and the second branch flow path, respectively, are caused to flow out from the flowing-out portion to the outside, no pressure difference is generated between the first branch flow path and the second branch flow path. Thus, the cells can be precisely sorted. Also, the cells sorted can be taken out from the cell holding portion to the outside.

Preferably, said cell holding portion may have a film-like portion adapted to be dug from an outside.

The cell holding portion has the film-like portion adapted to be dug from the outside. Therefore, a pipette, for example, is put in the cell holding portion through the film-like portion, which results in that the cells held in the cell holding portion can be simply taken out to the outside by using the pipette or the like.

According to still another embodiment of the present disclosure, there is provided a cell sorting method including: branching a flow path in which a fluid containing therein cells flows into a first branch flow path and a second branch flow path; coupling said first branch flow path and the second branch flow path to each other; and causing liquids flowing in the first branch flow path and the second branch flow path coupled to each other, respectively, to flow out to an outside.

In the embodiment of the present disclosure, the first branch flow path and the second branch flow path which branch once are coupled to each other again. Therefore, when the fluids flowing in the first branch flow path and the second branch flow path, respectively, are caused to flow out from the flowing-out portion to the outside, no pressure difference is generated between the first branch flow path and the second branch flow path. Thus, the cells can be precisely sorted.

As set forth hereinabove, according to the present disclosure, since the first branch flow path and the second branch flow path which branch once are coupled to each other again, no pressure difference is generated between the first branch flow path and the second branch flow path. Thus, the cells can be precisely sorted.

Additional features and advantages are described herein, and will be apparent from the following Detailed Description and the figures.

### BRIEF DESCRIPTION OF THE FIGURES

FIG. 1 is a conceptual view of a cell function analyzing/sorting system according to an embodiment of the present disclosure.

FIG. 2 is a perspective view showing a structure of a cell sorting chip which can be applied to the cell function analyzing/sorting system shown in FIG. 1.

FIG. 3 is a top plan view showing a structure of a sorting portion (a sorting signal is held in an OFF state) shown in FIG. 2.

FIG. 4 is a cross sectional view taken on line A-A of FIG. 3.

FIG. 5 is a top plan view showing the structure of the sorting portion (the sorting signal is held in an ON state) shown in FIG. 2.

FIG. 6 is a top plan view showing a structure of a cell holding portion and a flowing-out portion both shown in FIG. 2.

FIG. 7 is a cross sectional view taken on line A-A of FIG. 6.

FIG. 8 is a schematic cross sectional view of the cell holding portion shown in FIG. 6.

FIG. 9 is a view explaining an operation and effects relating to the present disclosure.

### DETAILED DESCRIPTION

Embodiments of the present application will be described below in detail with reference to the drawings.

(Outline of Cell Function Analyzing/Sorting System)

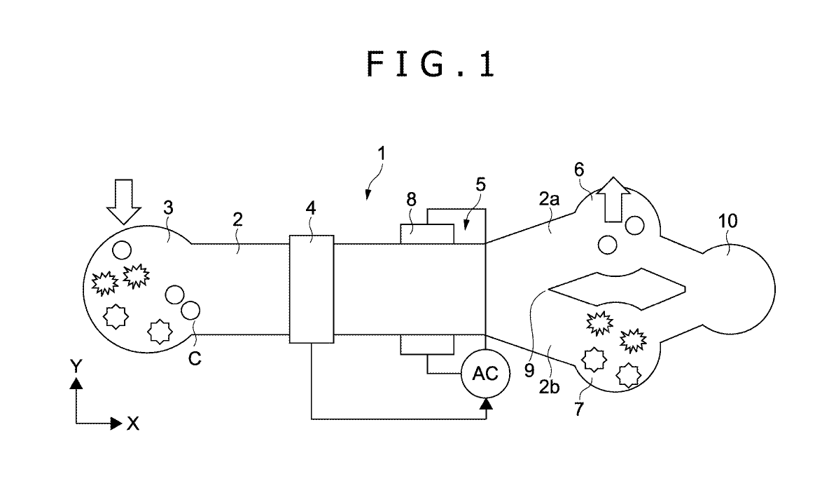

FIG. 1 is a conceptual view of a cell function analyzing/sorting system according to an embodiment of the present disclosure.

As shown in FIG. 1, the cell function analyzing/sorting system 1 is provided with a putting-in portion 3, a measuring portion 4, a sorting portion 5, cell holding portions 6 and 7, and a flowing-out portion 10 from an upstream side of a micro-flow path (hereinafter referred simply to as “a flow path”) 2 along the flow path. The sorting portion 5 includes an electric field applying portion 8 and a branch portion 9.

Reference symbol C designates a cell. A liquid containing therein the cells C sampled is put in a pressure container (not shown), for example, using a pump or the like.

The liquid put in from the putting-in portion 3 flows in the flow path 2. The flow path 2 branches into a branch flow path 2a and a branch flow path 2b at the branch portion 9. The branch flow path 2a and the branch flow path 2b branching once are coupled again in a flowing-out portion 10. Although the flowing-out portion 10 has a function as such a coupling portion, the coupling portion may be provided separately from the flowing-out portion 10. The cell holding portion 6 is provided in the middle of the branch flow path 2a and the cell holding portion 7 is provided in the middle of the branch flow path 2b.

The measuring portion 4 measures complex permittivities of the individual cells C flowing in the flow path 2 over multipoint frequencies (three points or more in frequency, typically, in the range of about 10 to about 20 points in frequency) in the frequency range (for example, in the range of 0.1 to 50 MHz) in which a dielectric relaxation phenomenon of the cells C occurs. The measuring portion 4 determines whether or not the cells C are cells to be sorted in accordance with the complex permittivities of the cells C thus measured. When it is determined that the cells C are cells to be sorted, the measuring portion 4 outputs a sorting signal. Instead of providing the measuring portion 4, a portion corresponding thereto, for example, may be composed of a signal detecting portion composed of a pair of electrodes, and a cell function analyzing portion for analyzing functions of the cells C in accordance with a signal detected.

The sorting portion 5 sorts the desired cells C of plural kinds of cells C put in from the putting-in portion 3 into the cell holding portion 6, and sorts the cells other than the desired cells C into the cell holding portion 7.

The electric field applying portion 8 can apply an electric field having a gradient in a direction different from an X direction in which the fluid flows, for example, in a Y direction perpendicular to the X direction. For example, the electric field applying portion 8 does not apply the electric field when the sorting signal is not inputted, but the electric field applying portion 8 applies the electric field when the sorting signal is inputted.

The branch portion 9 branches the cells C to which no electric field is applied in the electric field applying portion 8 in such a way that the cells C flow in the cell holding portion 7. Also, the branch portion 9 branches the cells C to which the electric field is applied in the electric field applying portion 8 in such a way that the cells C flow in the cell holding portion 6.

The liquids which have passed through the cell holding portions 6 and 7, respectively, are discharged from the flowing-out portion 10 to the outside through the pressure container, for example, using the pump. A pressure difference is generated in the flow path 2 due to application of a pressure in the putting-in portion 3, and reduction of a pressure in the flowing-out portion 10.

In the cell function analyzing/sorting system 1, the electric field is applied to the cells based on ON/OFF or amplitude modulation of the electric field in accordance with the sorting signal which is previously outputted either from a measuring portion or from a measured value analyzing portion by using some sort of another technique. Thus, even in the case of a cell group having a dispersion in cell diameter or physical property of the cells C, only the cells C as an object of the sorting are sorted based on a sufficient dielectrophoretic force.

(Chip for Analyzing/Sorting Cell Functions)

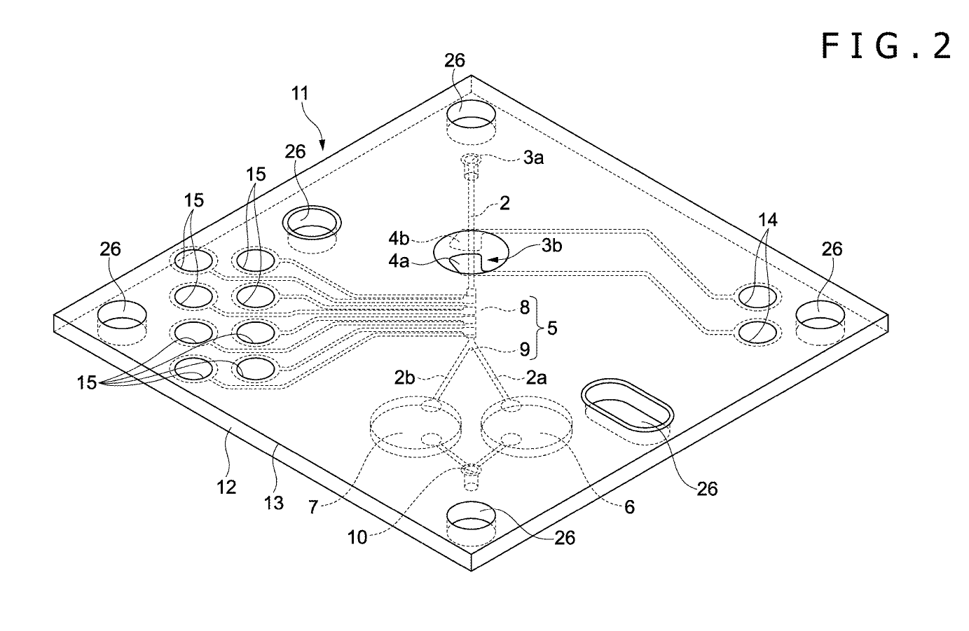

FIG. 2 is a perspective view showing a structure of a cell sorting chip which is applied to the cell function analyzing/sorting system shown in FIG. 1.

As shown in FIG. 2, a chip 11 includes a substrate 12 and a sheet-like member 13 made of a polymer film or the like. The substrate 12 is provided with the flow path 2, a liquid putting-in portion 3a as the putting-in portion 3, the branch portion 9, the cell holding portions 6 and 7, and the flowing-out portion 10. Trenches or the like are formed in a surface of the substrate 12, and surfaces thereof are covered with the sheet-like member 13, thereby structuring these constituent elements. The cell putting-in portion 3b in which the liquid containing therein the cells C is put is structured by providing a fine hole in the sheet-like member 13. When the liquid containing therein the cells C dropped on the cell putting-in portion 3b with a pipette, the liquid containing therein the cells C flows downstream with respect to the flow path 2 so as to be dragged in the liquid flowing in the flow path 2 through the fine hole. Because of the fine hole, plural cells C do not collectively flow into the flow path 2, but plural cells C flow into the flow path 2 one by one.

A pair of signal detecting electrodes 4a and 4b are provided so as to hold the fine hole between the signal detecting electrodes 4a and 4b. One signal detecting electrode 4a is provided on a front surface of the sheet-like member 13, and the other signal detecting electrode 4b is provided on a back surface of the sheet-like member 13. An electrode pair which will be described later and which composes the electric field applying portion 8 is also formed on the back surface of the sheet-like member 13.

Upper portions of the cell holding portions 6 and 7 are covered with the sheet-like member 13 as a film-like portion which can be dug from the outside. However, the pipette is stung to the sheet-like member 13, thereby making it possible to take out the cells C through the pipette.

Electrode pads 14 take out a signal detected by the signal detecting electrodes 4a and 4b to the outside. The signal thus taken out, for example, is sent to a cell function analyzing portion (not shown). The sorting signal outputted from the cell function analyzing portion is inputted to electrode pads 15. The sorting signal thus inputted is then sent to the electrode pair composing the electric field applying portion 8.

Through holes 26 are positioning holes when the chip 11 is mounted to an apparatus main body having the cell function analyzing portion and the like.

(Structure of Sorting Portion)

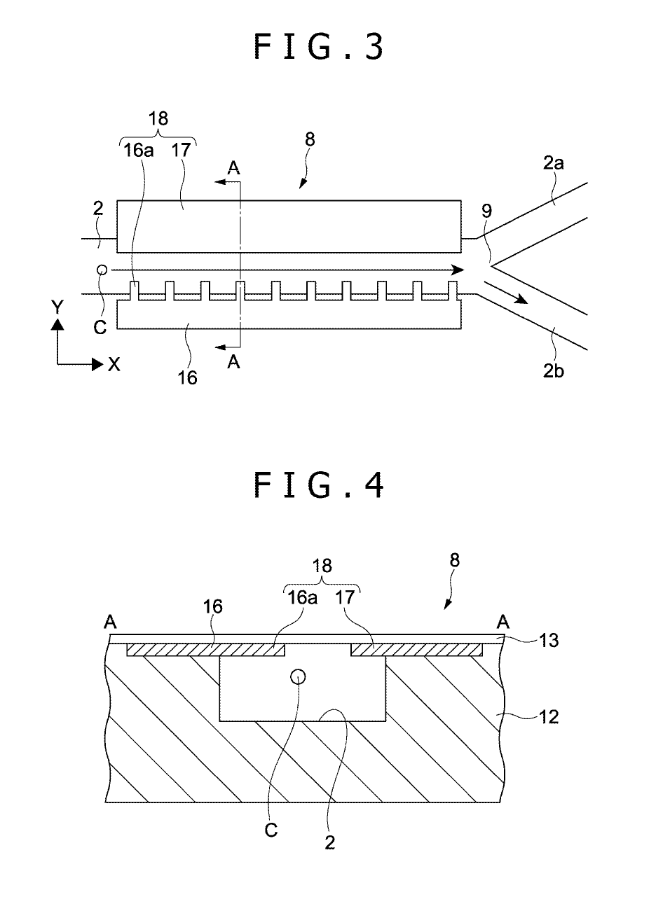

FIG. 3 is a top plan view showing a structure of the sorting portion 5 shown in FIG. 2, and FIG. 4 is a cross sectional view taken on line A-A of FIG. 3.

As shown in FIGS. 3 and 4, the sorting portion 5 includes the electric field applying portion 8 and the branch portion 9.

The electric field applying portion 8 includes electrodes 16 and 17 provided in predetermined positions of the flow path 2, respectively. The electrodes 16 and 17 are disposed so as to hold the flow path 2 between the electrodes 16 and 17 in a direction different from a direction (X direction) of the fluid flowing in the flow path 2, for example, in a Y direction and so as to face each other. The electrodes 16 and 17 are provided on a back surface (an upper surface within the flow path 2) of the sheet-like member 13. The electrode 16, for example, is an electrode to which a signal is applied and is structured in such a way that a large number of electrode fingers 16a protrude toward the electrode 17. The electrode 17, for example, is a common electrode and is structured so as not to have irregularities for the electrode 16. In the following description, a combination of one electrode finger 16a and the electrode 17 is referred to as an electrode pair 18. The electrode pair 18 is structured in such a way, whereby when the signal is applied across the electrodes 16 and 17, the electric fields which have gradients in the Y direction are applied across the electrode pairs 18, respectively.

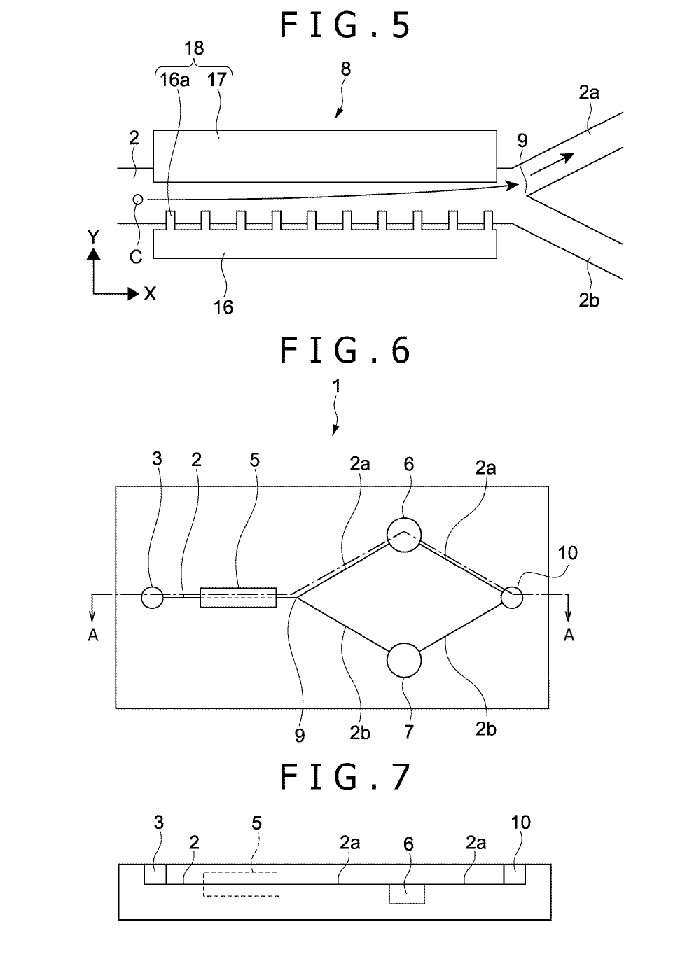

The branch portion 9 branches the cells C whose flow directions are changed by the dielectrophoretic force generated by application of the electric field by the electric field applying portion 8 into the branch flow path 2a and the branch flow path 2b in a predetermined position located downstream with respect to the electric field applying portion 8 within the flow path 2. The flow path 2 branches into a Y letter shape, thereby structuring the branch portion 9. One branch portion extends toward the cell holding portion 6 through the branch flow path 2a, and the other branch portion extends toward the cell holding portion 7 through the branch flow path 2b. For example, in the putting-in portion 3, the cells C are put in a biased position on the cell holding portion 7 side. With regard to the cells C put in the biased position on the cell holding portion 7 side in such a way, when the cells C which are not as an object of the sorting pass through the electric field applying portion 8, no electric field is applied to any of the cells in the electric field applying portion 8 (non-active). Thus, as shown in FIG. 3, the cells C concerned flow in the flow path 2 on the biased position side to flow in the cell holding portion 7 as they are. On the other hand, when the cells C which are as an object of the sorting pass through the electric field applying portion 8, the electric field is applied to the cells concerned in the electric field applying portion 8 (active), and thus the dielectrophoretic force is applied to the cells C. As a result, as shown in FIG. 5, the flow direction of the cells C is changed to the cell holding portion 6 side, and thus the cells C as the object of the sorting are branched to the cell holding portion 6 side by the branch portion 9.

In the electric field applying portion 8 structured in such a way, the electric fields which have the gradients in the Y direction in the electrode pairs 18, respectively, are applied. Therefore, the cells C passing through the electric field applying portion 8 can be gradually changed in the path thereof to branch to the cell holding portion 6 side.

(Structures of Cell Holding Portion and Flowing-out Portion)

FIG. 6 is a top plan view showing structures of the cell holding portion and the flowing-out portion, and FIG. 7 is a cross sectional view taken on line A-A of FIG. 6.

As shown in FIGS. 6 and 7, the cell holding portion 6 is provided in the middle of the branch flow path 2a, and the cell holding portion 7 is provided in the middle of the branch flow path 2b. A termination of the branch flow path 2a and a termination of the branch flow path 2b are coupled to each other again at the flowing-out portion 10. Therefore, the liquid flowing in the branch flow path 2a passes through the cell holding portion 6 so as to flow into the flowing-out portion 10, and the liquid flowing in the branch flow path 2b passes through the cell holding portion 7 so as to flow into the flowing-out portion 10.

Each of the cell holding portions 6 and 7 operates as a flow path abruptly expanding portion. Thus, the cells C do not flow into the flowing-out portion 10, but are held in the cell holding portions 6 and 7.

For example, each of the cell holding portions 6 and 7 is composed of a bottomed hole having a cylindrical shape which is sufficiently deeper in depth than each of the branch flow paths 2a and 2b, and is sufficiently larger in diameter than a width of each of the branch flow paths 2a and 2b.

As described above, in each of the cell holding portions 6 and 7, an average cross-sectional area of a surface vertical to the flow direction (X direction) of the liquid is sufficiently larger than a cross-sectional area of a surface of each of the branch flow paths 2a and 2b vertical to the flow direction. Therefore, a settling-out velocity, v, of each of the cells C becomes larger than a flow rate, u, of each of the cells C in the flow direction.

The cell holding portions 6 and 7 are suitably designed, which results in that although the fluid itself usually flows out from the flowing-out portion 10 to the container(s) in the outside of the apparatus, the cells C settling out in the cell holding portions 6 and 7 stay in recirculation regions or dead water regions generated in the cell holding portions 6 and 7, respectively, and thus do not flow out to the flowing-out portion 10. That is to say, in order to attain this, it is only necessary that a ratio between a length of each of the cell holding portions 6 and 7 and the average cross-sectional area of each of the cell holding portions 6 and 7, and the average cross-sectional area of each of the branch flow paths 2a and 2b is set in such a way that a distance by which the cells C settle out while the cells C pass through the cell holding portions 6 and 7 becomes larger than a height of each of the branch flow paths 2a and 2b.

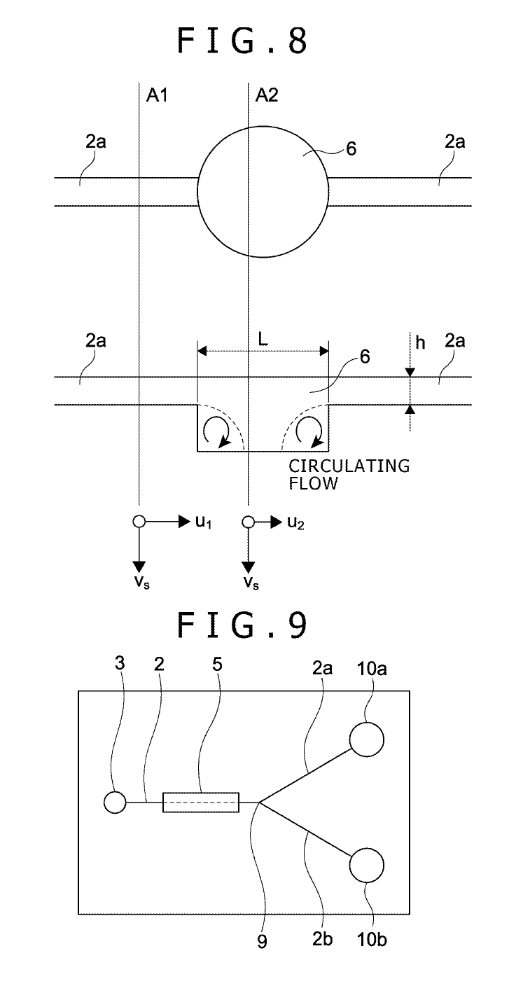

Here, as shown in FIG. 8, a time for which the cells C pass through the cell holding portion 6, 7 is given by t, an average flow rate of the cells C within the branch flow path 2a, 2b in a mainstream direction (X direction) is given by u₁, an average flow rate in a height direction (Z direction (a gravity direction is set as positive)) is given by v₁, an average flow rate of the cells C within the cell holding portion 6, 7 in the mainstream direction (X direction) is given by u₂, and an average flow rate in the height direction (Z direction (the gravity direction is set as positive)) is given by v₂. In addition, with regard to a surface vertical to the mainstream direction (X direction), an average cross-sectional area of the branch flow path 2a, 2b is given by A₁, an average cross-sectional area of the cell holding portion 6, 7 is given by A₂, a flow path height within the branch flow path 2a, 2b is given by h, and a mainstream direction length of the cell holding portion 6, 7 is given by L.

A settling-out velocity in the gravity direction is obtained from the Stokes equation, more strictly, from a drag acting on a ball in a uniform stream. When the settling-out velocity in the gravity direction is given by vₛ, a relationship of v₁=v₂=vₛ is obtained because vₛ does not depend on the flow rate in the mainstream direction.

Now, a time, t, required for the cells C to pass through the cell holding portion 6, 7 is expressed by Expression (1):

t=L/u₂=(L/u₁)×(A₂/A₁) (1)

At this time, a distance, zₛ, by which the cells C settle out for the time, t, is expressed by Expression (2):

zₛ=vₛ×t (2)

when zₛ is larger than the flow path height, h, the cells C are trapped in a circulating flow or the dead water region existing below the cell holding portion 6, 7, and thus stay in the cell holding portion 6, 7 without flowing into the flowing-out portion 10. That is to say, it is only necessary for this condition to fulfill Conditional Expression (3):

zₛ=vₛ×t>h (3)

A description will be further given below by using concrete numerical values.

As an example of the cells C, a specific gravity, ρ_{cell}, of a white blood cell is in the range of 1.063 to 1.085, and a typical value thereof is given by 1.07. Also, a diameter of the cell C is given by 10 μm, in a word, a radius, r, of the cell C is given by 5 μm.

A density, ρ_{H2O}, of water as an example of a fluid is given by 1,000 kg/cm³ at 20° C., and a viscosity coefficient, μ, of the water is given by 0.001 Pa·S at 20° C.

In this case, the settling-out velocity, vₛ, of the cell C in the gravity direction is given from the Stokes equation by: vₛ=2/9×(ρ_{cell}−ρ_{H2O})gr₂/μ=2.2 (μm/s)

In addition, the settling-out velocity, vₛ, of the cell C in the gravity direction is given from a general drag equation for the ball by: vₛ=3.8 (μm/s)

Here, when a mainstream direction length L of the cell holding portion 6, 7 is L=5 (nm), the ratio Sᵣ (A₁:A₂) between the average cross-sectional area A₁ of the branch flow path 2a, 2b, and the average cross-sectional area A₂ of the cell holding portion 6, 7 in the mainstream direction (X direction) is Sᵣ=100, and the average flow rate u₁ of the cell C within the branch flow path 2a, 2b in the mainstream direction (X direction) is u₁=10 (μm/s), the flow path height, h, within the branch flow path 2a, 2b is h =50 (μm).

Also, when the time, t, for which the cell C passes through the cell holding portion 6, 7 is t=50 (s), the distance, zₛ, by which the cell C settles out for the time, t, is given by zₛ=vₛ×t=3.8 (μm/s)×50 (s)=190 (μm).

Since this calculation result fulfills a relationship of zₛ>h, the cell C is trapped in the circulating flow or the dead water region existing below the cell holding portion 6, 7, and thus stay in the cell holding portion 6, 7 without flowing into the flowing-out portion 10. In addition, even when a dispersion of the cell diameter is set to ±2 (μm) and the calculation is similarly carried out with respect to the minimum diameter cell, a relationship of zₛ=130 (μm)>h is obtained. Thus, in the case as well, the cell C is trapped in the circulating flow or the dead water region existing below the cell holding portion 6, 7, and thus stay in the cell holding portion 6, 7 without flowing into the flowing-out portion 10.

From the foregoing, when both of the practical size of the chip 11, and the sorting speed used in the apparatus are taken into consideration, it is understood that an achievable design resolution exists.

(Cell Sorting Method)

A cell sorting method according to another embodiment of the present disclosure includes: branching the flow path 2 in which the fluid containing therein the cells C flows into the branch flow path 2a and the branch flow path 2b; coupling the branch flow path 2a and the branch flow path 2b to each other; and causing the liquid flowing in the branch flow path 2a and the branch flow path 2b coupled to each other, respectively, to flow out to the outside.

(Operation and Effects)

As shown in FIG. 9, for the purpose of carrying out the intra-flow path sorting for the cells C based on some sort of cell sorting information, the apparatus needs to include at least the sorting portion 5 within the flow path 2, and the branch flow paths 2a and 2b, and the flowing-out portions 10a and 10b which are all located downstream with respect to the sorting portion 5. In addition, it is necessary to previously realize the stable liquid sending.

With a method of sending the liquid within the flow path based on the pressure difference by using the pressure container using the pump or the like, since the fluid can be isolated from the outside atmospheric pressure by adopting the sealed structure, the stable liquid sending can be carried out as compared with the liquid sending by using the pump in which the pulsation essentially exists. In this case, with regard to the fluid flowing out from the branch portion 9 into the flowing-out portions 10a and 10b, a static pressure in each of the flowing-out portions 10a and 10b needs to be stable, and the static pressures in the flowing-out portions 10a and 10b need to be equal to each other, or needs to be usually, strictly kept at a desired ratio. The reason for this is because when the static pressures in the flowing-out portions 10a and 10b are changed, the amounts of fluids which flow from the mainstream into the branch flow paths 2a and 2b through the branch portion 9 are changed in accordance with a ratio in difference pressure between the static pressure in the branch portion 9, and the static pressure in each of the flowing-out portions 10a and 10b. When the amounts of fluids are changed, the branch flow paths 2a and 2b into which the cells flow are readily changed, so that even when how a driving force for the sorting is applied either to the cells themselves or to the entire fluid containing therein the cell C short of the branch portion 9, it may be impossible to send the cells C to the desired flowing-out portion 10a, 10b no longer.

Then, in the embodiments of the present disclosure, when the pressure driving is carried out, the flowing-out portions are coupled to each other, and the fluid is discharged into the low-pressure side pressure container in the outside, which results in that the stable liquid sending free from the pressure change becomes possible. That is to say, according to the embodiments of the present disclosure, no pressure difference is generated between the branch flow paths 2a and 2b, and thus it is possible to readily realize the stable liquid sending which is essential to the cell sorting within the flow path 2. In addition, the cells C after the sorting stay in the cell holding portions 6 and 7 within the chip 11, and thus it is unnecessary to specially prepare the pipe and the container, to say nothing of causing the cells C to fly in the air. Therefore, it is possible to readily and inexpensively realize the contamination-free and thus it is possible to apply the present disclosure to the regenerative medicine.

It is noted that the present disclosure is by no means limited to the embodiments described above, and various kinds of changes can be made within the range of the technical idea.

For example, although in the embodiments described above, the case where the coupling portion and the flowing-out portion are integrated with each other is exemplified, the coupling portion and the flowing-out portion may also exist separately from each other. For example, the two branch flow paths may be coupled to each other once at the coupling portion to be connected to the flowing-out portion through the common flow path.

Although in the embodiments described above, the case where the two branch flow paths are provided is exemplified, three or more branch flow paths may also be provided.

Although in the embodiments described above, the case where the cell holding portion is provided in each of the branch flow paths is shown, a structure may also be adopted such that the cell holding portion is provided only in the branch flow path for sorting the cells.

Although the shape, size and the like of each of the cell holding portions are by no means limited to these in the embodiments described, of course, each of the cell holding portions can be embodied in various forms.

It should be understood that various changes and modifications to the presently preferred embodiments described herein will be apparent to those skilled in the art. Such changes and modifications can be made without departing from the spirit and scope and without diminishing its intended advantages. It is therefore intended that such changes and modifications be covered by the appended claims.

The application is claimed as follows:

1. A cell sorting apparatus, comprising:
a branch portion branching a flow path in which a fluid containing therein cells flows into a first branch flow path, and a second branch flow path;
a coupling portion coupling said first branch flow path and said second branch flow path to each other; and
a flowing-out portion causing liquids flowing in said first branch flow path and said second branch flow path coupled to each other by said coupling portion, respectively, to flow out to an outside.

2. The cell sorting apparatus according to claim 1, further comprising:
a cell holding portion provided in said first branch flow path and holding the cells,
wherein a length of said cell holding portion, and a ratio between an average cross-sectional area of said cell holding portion and an average cross-sectional area of said first branch flow path are set in such a way that a distance by which the cells settle out while the cells pass through said cell holding portion becomes larger than a height of said first branch flow path.

3. A cell sorting chip, comprising:
a substrate;
a flow path which is provided in said substrate and in which a fluid containing therein cells flows;
a first branch flow path and a second branch flow path provided in said substrate and branching at said flow path;
a cell holding portion provided in said first branch flow path and holding the cells contained in the fluid flowing in said first branch flow path; and
a flowing-out portion coupling said first branch flow path and said second branch flow path to each other and causing liquids flowing in said first branch flow path and said second branch flow path to flow out to an outside.

4. The cell sorting chip according to claim 3, wherein said cell holding portion has a film-like portion adapted to be dug from an outside.

5. A cell sorting method, comprising:
branching a flow path in which a fluid containing therein cells flows into a first branch flow path and a second branch flow path;
coupling said first branch flow path and said second branch flow path to each other; and
causing liquids flowing in said first branch flow path and said second branch flow path coupled to each other, respectively, to flow out to an outside.
